(19) United States
(12) Patent Application Publication
(10) Pub. No.: US 2006/0128984 A1
(43) Pub. Date: Jun. 15, 2006

(54) Method of preparing an ester of an aromatic carboxylic acid bearing at least one perfluoroalkyl group

(76) Inventors: Alain Chenede, Vienne (FR); Laurent Saint-Jalmes, Vourles (FR); Philippe-Jean Tirel, Communay (FR)

Correspondence Address: Jean-Louis Seugnet;Intellectual Property Dept Rhodia, 259 Prospect Plains Road, CN 7500, Cranbury, NJ, 08512, US

(21) Appl. No.: 10/521,825

(22) PCT Filed: Jul. 23, 2003
(86) PCT No.: PCT/FR03/02333

§ 371 Date: Aug. 15, 2005

(30) Foreign Application Priority Data
Jul. 25, 2002 (FR) 02 09443

(51) Int. Cl. C07C 69/76

(52) U.S. Cl. 560/103

(57) ABSTRACT

The invention relates to a method of preparing an ester of an aromatic carboxylic acid bearing at least one perfluoroalkyl group on the aromatic ring and, more specifically, alkyl esters with low carbon condensation. The inventive method is characterised in that it consists in (i) reacting an aromatic compound bearing at least one perfluoroalkyl group and at least one nitrile group on the aromatic ring, an alkanol and a strong protonic acid at a temperature of at least 45° C. and (ii) recovering the ester thus produced.

The subject of the present invention is a method for preparing an ester of an aromatic carboxylic acid bearing at least one perfluoroalkyl group on the aromatic ring and more particularly a trifluoromethyl group.

The invention relates in particular to alkyl esters with low carbon condensation.

It relates most particularly to the preparation of methyl(trifluoromethyl)benzoates.

There are several routes for synthesizing (trifluoromethyl)benzoic acid esters starting with various raw materials.

Thus, there is described in J. Org. Chem. (1989), 54(13), 3062-3068, a method of preparation which consists in a first step of preparing a magnesium compound from 3-bromotrifluoromethylbenzene and then its condensation with carbon dioxide, followed by acidification in order to obtain 3-(trifluoromethyl)benzoic acid and direct esterification with methanol, in the presence of an acid catalyst. The yield obtained is 70%.

However, said method has the disadvantage of involving the magnesium compound of 3-bromo(trifluoro-methyl)benzene which is unstable and which can lead to explosions (see Chemistry and Industry of 23 Jan. 1971, p. 120).

It is also known to prepare the methyl ester of (trifluoromethyl)benzoic acid by reacting carbon monoxide and methanol with bromo(trifluoro-methyl)benzene) but this method requires either electrochemistry [J. Electroanal. Chem (1994), 379(1-2), 111-120], or palladium-type metal catalysis [Synth. Comm. (1993), 23(10), 1361-1370], and is therefore particularly expensive.

The problem posed is that the manufacture of the aromatic carboxylic acid, a precursor of the ester, is difficult to carry out.

The objective of the present invention is to provide a method which overcomes the abovementioned disadvantages and which allows access to esters according to any other route.

There has now been found, and that is the subject of the present invention, a method for preparing an ester of an aromatic carboxylic acid bearing at least one perfluoroalkyl group on the aromatic ring according to the invention is characterized in that it consists in reacting an aromatic compound bearing at least one perfluoroalkyl group and at least one nitrile group on the aromatic ring, an alkanol and a strong protonic acid, at a temperature of at least 45° C., and then in recovering the ester obtained.

There is used in the method of the invention an aromatic compound bearing at least one nitrile group and at least one perfluoroalkyl group corresponding to general formula: 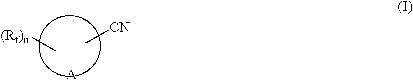
in said formula (I):
A represents the residue of a benzene or naphthalene ring,
R_{f} represents a perfluoroalkyl chain of formula —[CF₂]ₚ—CF₃ in which p represents a number ranging from 0 to 10,
n is a number at least equal to 1, preferably between 1 and 3.

The substrates preferably used in the method of the invention correspond to formula (I) in which R_{f} represents a group —CF₃ or —CF₂—CF₃, preferably a trifluoromethyl group and n is a number equal to 1 or 2.

The invention relates more particularly to an aromatic compound bearing at least one nitrile group and at least one trifluoromethyl group corresponding to general formula: 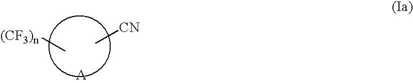
in said formula (Ia):
A represents the residue of a benzene or naphthalene ring,
n is a number at least equal to 1, preferably between 1 and 3.

In accordance with the method of the invention, the ester is directly obtained starting with a nitrile-type compound as long as the reaction is carried out at a temperature of at least 45° C.

As is demonstrated in the examples, there is no formation of ester but of the iminoester hydrochloride when the temperature is less than 45° C., which necessarily involves an additional step of hydrolysis in order to obtain the ester.

There is used in the method of the invention an aromatic compound bearing at least one nitrile group and at least one perfluoroalkyl group, preferably trifluoromethyl corresponding to general formula (I).

In the remainder of the text, said compound is designated by the expression “nitrile-type compound”.

The substrates preferably used in the method of the invention correspond to formula (Ia₁) in which: 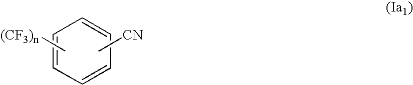
in said formula (Ia₁):
n is a number at least equal to 1, preferably between 1 and 3,
at least one trifluoromethyl group is at a 3-, 4- or 5-position.

If the compound of formula (Ia₁) comprises a single trifluoromethyl group (n=1), the latter is advantageously at the 3- or 4-position.

If the compound of formula. (Ia₁) comprises two trifluoromethyl groups (n=2), the latter are preferably at the 3- and 4- or 3- and 5-position.

The invention does not exclude the presence of other substituents on the aromatic ring as long as they do not interfere with the reaction. There may be mentioned in particular alkyl or alkoxy groups, nitro groups or halogen atoms, chlorine or bromine.

Preferably, a starting substrate is used which has good purity. Thus, it is preferable to use a compound comprising less than 10% of water because the presence of water in the medium slows the reaction kinetics.

In accordance with the method of the invention, the nitrile-type compound is reacted with an alkanol which can be symbolized by the following formula:
R₁—OH (II)
in said formula (II), R₁ represents an alkyl, cycloalkyl or arylalkyl group.

In the context of the invention, the expression “alkyl” is understood to mean a linear or branched hydrocarbon chain having from 1 to 15 carbon atoms and preferably 1 or 4 carbon atoms.

The expression “cycloalkyl” is understood to mean a cyclic hydrocarbon group comprising from 3 to 8 carbon atoms, preferably a cyclopentyl or cyclohexyl group.

The expression “arylalkyl” is understood to mean a linear or branched hydrocarbon group bearing a monocyclic aromatic ring and comprising from 7 to 12 carbon atoms, preferably benzyl.

The term “alkanol” will be used hereinafter in a generic manner to designate any alkanols corresponding to formula (II).

The method is more particularly used for a low aliphatic alcohol having from 1 to 4 carbon atoms, such as for example methanol, ethanol, trifluoroethanol, propanol, isopropyl alcohol, butanol, isobutyl alcohol, sec-butyl alcohol, tert-butyl alcohol; a cycloaliphatic alcohol having from 3 to about 8 carbon atoms, such as for example cyclopentanol, cyclohexanol; an aliphatic alcohol bearing an aromatic group having from 7 to about 20 carbon atoms such as for example benzyl alcohol and phenethyl alcohol.

Among the abovementioned alcohols, methanol or ethanol are preferably used in the method of the invention.

The quantity of alkanol used is at least equal to the stoichiometric quantity for the reaction.

Generally, the alkanol is used in a quantity such that the ratio between the number of moles of alkanol and the number of moles of nitrile-type compound (I) varies between about 1 and about 20, preferably between 2 and 6.

One of the advantages of the method of the invention is to be able to have access to the ester in concentrated medium. Indeed, the concentration of the nitrile-type compound in the medium may be at least 50% by weight, preferably between 70 and 85% by weight.

It should be noted that the method of the invention does not exclude the use of an organic solvent.

A condition which determines the choice of the solvent is that it should not react under the reaction conditions.

A second requirement is that it should satisfy certain basicity characteristics. Indeed, said solvent should not be too basic. To determine if a solvent satisfies this requirement, its basicity is assessed with reference to the “donor number”. A polar organic solvent having a donor number of less than or equal to 10 is chosen. The lower limit is not of a critical nature. An organic solvent having a donor number of between 2 and 10 is preferably chosen.

It is recalled that the “donor number”, abbreviated DN, gives an indication of the nucleophilic character of the solvent and reveals its capacity to give its doublet.

The book by Christian REICHARDT [Solvents and Solvent Effects in Organic Chemistry—VCH p. 19 (1988)] gives the definition of “donor number” which is defined as the negative (−ΔH) of the enthalpy (Kcal/mol) of the interaction between the solvent and antimony pentachloride, in a dilute solution of dichloroethane.

As examples of organic solvents satisfying the abovementioned basicity characteristics, which can be used in the method of the invention, there may be mentioned more particularly aromatic ether oxides and, more particularly diphenyl ether.

There may also be mentioned halogenated or non-halogenated, aliphatic, cycloaliphatic or aromatic hydrocarbons.

By way of examples of aliphatic hydrocarbons, there may be mentioned more particularly paraffins such as in particular hexane, cyclohexane, methylcyclohexane, petroleum cuts of the petroleum ether type. There may be mentioned more particularly aromatic hydrocarbons such as in particular benzene, toluene, xylenes, cumene, petroleum cuts consisting of a mixture of alkylbenzenes, in particular Solvesso-type cuts.

As examples more particularly of organic solvents, there may be mentioned aliphatic halogenated hydrocarbons and more particularly n-chlorobutane, dichloromethane, 1,2-dichloroethane; aromatic halogenated hydrocarbons, and more particularly mono- or dichlorobenzene.

It is also possible to use a mixture of solvents.

The quantity of organic solvent to be used may be variable. It can be as large as that of the reagents. Thus, the solvent may represent from 10 to 50% of the weight of the reaction medium.

In accordance with the method of the invention, the nitrile-type compound and the alkanol are reacted in the presence of a strong protonic acid.

A strong protonic acid is used in the method of the invention.

The expression strong acid denotes, in the present invention, a protonic acid having a pKa in water of less than that of the starting nitrile-type compound.

The pKa is defined as the ionic dissociation constant of the acid/base pair when water is used as solvent.

As non limiting examples of strong protonic acids, there may be mentioned more particularly hydracids such as hydrochloric acid, hydrobromic acid; halogenated or nonhalogenated oxyacids such as sulfuric acid, perchloric acid; halogenated or nonhalogenated sulfonic acids such as fluorosulfonic acid, chlorosulfonic acid or trifluoromethanesulfonic acid, methanesulfonic acid, ethanesulfonic acid, ethanedisulfonic acid, benzenesulfonic acid, benzenedisulfonic acids, toluenesulfonic acids, xylenesulfonic acids, naphthalenesulfonic acids and naphthalenedisulfonic acids; halocarboxylic acids such as in particular trichloroacetic acid.

Among these acids, hydrochloric acid, sulfuric acid, trifluoromethanesulfonic acid and methanesulfonic acid are preferably used.

Concentrated acid solutions are preferably used because the presence of water in the medium slows the reaction kinetics.

For example, use is made of a hydrochloric acid solution at 37% by weight, and of a sulfuric acid solution of at least 95%, preferably greater than 98%.

Advantageously, hydrochloric acid in gaseous form is used.

The quantity of acid, expressed as the ratio of the number of proton equivalents to the number of moles of nitrile-type compound can vary between about 1 and about 10, and preferably between 2 and 4.

The method of the invention is generally carried out under atmospheric pressure but preferably under a controlled atmosphere of inert gases. It is possible to establish an atmosphere of rare gases, preferably argon, but it is more economical to use nitrogen.

The invention may be carried out according to several embodiments.

A first variant consists in loading the nitrile-type compound and the alkanol, optionally an organic solvent, and then in introducing the acid, preferably bubbling of hydrochloric acid.

One of the characteristics of the method of the invention is the choice of the reaction temperature.

The latter is at least 45° C. The upper limit for the temperature is preferably less than the boiling point of the alkanol.

The expression “less” is generally understood to mean two or three degrees less.

In the case of a lower alcohol such as methanol or ethanol, the temperature is advantageously chosen between 45° C. and 60° C., preferably between 50° C. and 60° C.

For a heavier alcohol, the critical lower limit is greater than 45° C. but preferably corresponds to the temperature at which the alkyl halide appears for example in the case where hydrochloric acid is used.

It should be noted that the higher the chosen temperature, the higher the yield.

The acid is added gradually, continuously or in fractions over a period preferably varying between 3 and 8 hours.

At the end of the reaction, the ester is obtained in the reaction medium which can be recovered in a conventional manner. It is possible to add water so that the salts formed are solubilized and the acid obtained remains in the organic phase.

The reaction medium is allowed to separate by decantation and then the organic and aqueous phases are separated.

The product obtained is in the organic phase and it can be optionally extracted with an immiscible organic solvent and there may be mentioned in particular toluene and monochlorobenzene.

It can be represented by formula (III): 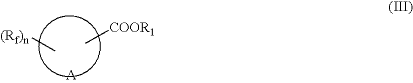
in said formula (III):
A, R_{f}, R₁ and n have the meaning given above.

According to another variant of the method of the invention and which is preferred, the nitrile-type compound and the alkanol are reacted as described above, optionally in the presence of an organic solvent, and then the acid is introduced, preferably bubbling of hydrochloric acid.

As mentioned above, the acid is added gradually and for a period of between 3 and 8 hours.

In a subsequent step, the water formed during the reaction is removed by distillation. It is possible for the water to be accompanied by excess alcohol depending on its boiling point.

To this effect, the distillation is carried out at a temperature of between 40° C. and 120° C., at atmospheric pressure and under reduced pressure ranging from 10 mm of mercury to atmospheric pressure, preferably between 10 and 200 mm of mercury.

In order to improve the yield, the alkanol and the acid are again added but in smaller quantities.

Thus, the alkanol is used in a quantity such that the ratio between the number of moles of alkanol and the number of moles of nitrile-type compound (I) varies between about 1 and about 3.

As for the acid, the quantity of acid, expressed as a ratio of the number of proton equivalents to the number of moles of nitrile-type compound, varies between about 1 and about 2.

After keeping at a temperature chosen between 45° C. and a temperature below the boiling point of the alkanol, and preferably about 60° C., for a period of 3 to 8 hours, the ester corresponding to formula (III) is obtained.

The ester obtained may be separated in a conventional manner and in particular as described above, that is to say addition of water, separation of the organic and aqueous phases and optionally extraction in an appropriate organic solvent.

The method of the invention makes it possible to obtain the esters with a high reaction kinetics, a high rate of conversion and a high reaction yield.

The present method is most particularly suitable for use continuously.

An exemplary embodiment of the invention is given below by way of illustration and without limitation.

In the examples, the abbreviations used are defined thus:

The rate of conversion (RC) corresponds to the ratio between the number of substrates converted and the number of moles of substrate used.

The actual yield (AY) corresponds to the ratio between the number of moles of product formed and the number of moles of substrate used.

### EXAMPLE 1

The following are loaded into a 3 liter glass reactor provided with central stirring, a condenser, a device for introducing a gas and a thermometer:
1027 g (6.00 mol) of m-trifluoromethylbenzo-nitrile,
and 576 g (18.0 mol) of dry methanol.

The medium is stirred, and then there is introduced, at room temperature, through an immersed tube, from a bottle on a balance, gaseous hydrochloric acid at a flow rate of 110 g/h (3 m/h) for 2 hours, that is 219 g (6.0 mol).

The reaction medium is heated to 60° C.

After having introduced the gaseous hydrochloric acid for 2 h, its introduction is further continued, reducing the flow rate by 2: that is 219 g (6.0 mol) over 4 hours.

The temperature is still kept at 60° C. for this introduction and then kept for 1 hour.

The analysis of the crude reaction material by gas chromatography gives RC(m-trifluoromethylbenzonitrile) of the order of 85%, AY(methyl 3-(trifluoro-methyl)benzoate) of the order of 75%.

After maintaining thus, the reactor is placed under a reduced pressure of 100 mm of mercury in order to distill the methanol and the water at a temperature of 70-75° C. The volume of the distillate is 350 ml.

576 g of dry methanol are added to the concentrate obtained after distillation and then gaseous hydrochloric acid is introduced using the same protocol, that is 219 g of dry gaseous hydrochloric acid over 2 hours, while allowing the temperature to rise up to 60° C. and maintaining this value.

The reaction requires being maintained for 3 hours at 60° C.

Once the reaction is complete, the reaction mass is cooled to 40° C. and then water (1000 g) is added in order to solubilize the ammonium chloride, and the aqueous and organic layers are separated by decantation.

The organic layer is washed with aqueous sodium bicarbonate (1%) in order to neutralize it.

GC analysis then gives a RC(m-trifluoromethylbenzo-nitrile)>98%, an AY(methyl 3-(trifluoro-methyl)benzoate)>95%.

### EXAMPLE 2

The following are loaded into a reactor:
1027 g (6.00 mol) of m-trifluoromethylbenzo-nitrile, p1 576 g (18.0 mol) of dry methanol.

The medium is stirred, and then there is introduced, at room temperature, through an immersed tube, from a bottle on a balance, gaseous hydrochloric acid at a flow rate of 110 g/h for 3 hours, that is 219 g (6.0 mol), while maintaining the temperature at 20-25° C.

After maintaining for 5 hours at 20-25° C., followed by hydrolysis of the reaction medium, analysis is carried out by gas chromatography which shows a rate of conversion of m-trifluoromethylbenzonitrile of 70% and a yield of methyl 3-(trifluoromethyl)benzoate of 42%.

### COMPARATIVE EXAMPLE 3

Example 1 is repeated but maintaining a temperature of 40° C. for 6 hours.

There is formation of only traces of methyl 3-(trifluoromethyl)benzoate.

The predominant product formed in an amount of 90% is the iminoester hydrochloride of formula:

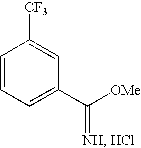

The latter precipitates from the medium and can therefore be separated by filtration.

1-31. (canceled)

32. A method for preparing an ester of an aromatic carboxylic acid bearing at least one perfluoroalkyl group on the aromatic ring, comprising the steps of:
a) reacting an aromatic compound bearing at least one perfluoroalkyl group and at least one nitrile group on the aromatic ring, an alkanol and a strong protonic acid, at a temperature of at least 45° C., and
b) recovering the ester obtained from the reaction medium of step a).

33. The method as claimed in claim 32, wherein the aromatic compound bearing at least one nitrile group and at least one perfluoroalkyl group corresponds to general formula: 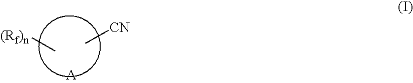
wherein:
A represents the residue of a benzene or naphthalene ring,
R_{f} represents a perfluoroalkyl chain of formula —[CF₂]ₚ—CF₃ in which p
represents a number ranging from 0 to 10, and
n is a number at least equal to 1.

34. The method as claimed in claim 33, wherein the aromatic compound bearing at least one nitrile group and at least one perfluoroalkyl group corresponds to general formula: 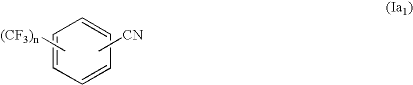
wherein:
n is a number at least equal to 1, and
at least one trifluoromethyl group is at a 3-, 4- or 5-position.

35. The method as claimed in claim 34, wherein n is equal to 1 and the trifluoromethyl group is at the 3- or 4-position or n is equal to 2 and the two trifluoromethyl groups are at the 3- and 4- or 3- and 5-position.

36. The method as claimed in claim 32, wherein the aromatic compound bearing at least one nitrile group is further substituted, with alkyl, alkoxy, nitro group or halogen atom.

37. The method as claimed in claim 36, wherein the aromatic compound bearing at least one nitrile group and at least one perfluoroalkyl group is m-trifluoromethylbenzonitrile.

38. The method as claimed in claim 32, wherein the alkanol corresponds to the following formula:
R₁—OH (II)
wherein, R₁ represents an alkyl, cycloalkyl or arylalkyl group.

39. The method as claimed in claim 38, wherein the alkanol is methanol or ethanol.

40. The method as claimed in claim 33, wherein the alkanol is used in a quantity such that the ratio between the number of moles of alkanol and the number of moles of aromatic compound bearing at least one nitrile group of formula (I) is between about 1 and about 20.

41. The method as claimed in claim 32, wherein the concentration of the aromatic compound bearing at least one nitrile group in the medium is at least 50% by weight.

42. The method as claimed in claim 32, wherein an organic solvent is further used in step a).

43. The method as claimed in claim 32, wherein a protonic acid having a pKa in water less than that of the starting nitrile-type compound is further used in step a).

44. The method as claimed in claim 43, wherein the strong protonic acid is a hydracid, halogenated oxyacid, nonhalogenated oxyacid, halogenatedsulfonic acid, nonhalogenated sulfonic acid, ethanedisulfonic acid, benzenesulfonic acid, benzenedisulfonic acid, toluenesulfonic acid, xylenesulfonic acid, naphthalenesulfonic acid, naphthalenedisulfonic acid, or halocarboxylic acid.

45. The method as claimed in claim 33, wherein the strong protonic acid is hydrochloric acid, sulfuric acid, trifluoromethanesulfonic acid or methanesulfonic acid.

46. The method as claimed in claim 33, wherein the hydrochloric acid is in gaseous form.

47. The method as claimed in claim 33, wherein the strong protonic acid is used in a quantity, expressed as the ratio of the number of proton equivalents to the number of moles of aromatic compound bearing at least one nitrile group, is between about 1 and about 10.

48. The method as claimed in claim 32, wherein the method is carried out under a controlled atmosphere of inert gases.

49. The method as claimed in one of claims claim 32, wherein the aromatic compound bearing at least one nitrile group and the alkanol, and, optionally, an organic solvent, are loaded, and then the acid is added.

50. The method as claimed in claim 32, wherein the reaction is carried out at a temperature of between 45° C. and a temperature less than the boiling point of the alkanol.

51. The method as claimed in claim 50, wherein the reaction temperature is between 50° C. and 60° C. and the alkanol is methanol or ethanol.

52. The method as claimed in claim 51, wherein the acid is added gradually, continuously or in fractions.

53. The method as claimed in claim 50, wherein, in step a) water is further added, the reaction medium is allowed to separate by decantation and, then, the organic and aqueous phases are separated.

54. The method as claimed in claim 53, wherein the ester obtained is extracted with the aid of an organic solvent from the organic phase.

55. The method as claimed in claim 50, wherein the water formed during the reaction is removed by distillation.

56. The method as claimed in claim 55, wherein the water is removed by distillation at a temperature of between 40° C. and 120° C., at atmospheric pressure or under reduced pressure ranging from 10 mm of mercury to atmospheric pressure.

57. The method as claimed in claim 55, wherein the alkanol and the acid are again added.

58. The method as claimed in claim 57, wherein the alkanol is used in a quantity such that the ratio between the number of moles of alkanol and the number of moles of the aromatic compound bearing at least one nitrile group is between about 1 and about 3.

59. The method as claimed in claim 57, wherein the acid is used in a quantity such that the ratio of the number of proton equivalents to the number of moles of the aromatic compound bearing at least one nitrile group is between about 1 and about 2.

60. The method as claimed in claim 57, wherein after maintaining at a temperature chosen between 45° C. and a temperature less than the boiling point of the alkanol in step a), the ester obtained is recovered in step b).

61. The method as claimed in claim 32, wherein the ester obtained is a methyl(trifluoromethyl)benzoate.

62. The method as claimed in claim 61, wherein the ester obtained is methyl 3 -(trifluoromethyl)benzoate.
